(19) United States
(12) Patent Application Publication
(10) Pub. No.: US 2009/0069584 A1
(43) Pub. Date: Mar. 12, 2009

(54) Levonorgestrel Crystallization

(75) Inventors: Yu-Sheng Chang, Jiali Town (TW); Shu-Ping Chen, Kaohsiung City (TW)

Correspondence Address: COHEN, PONTANI, LIEBERMAN & PAVANE LLP, 551 FIFTH AVENUE, SUITE 1210, NEW YORK, NY, 10176, US

(73) Assignee: ScinoPharm Taiwan Ltd., Tainan County (TW)

(21) Appl. No.: 12/231,751

(22) Filed: Sep. 5, 2008

Related U.S. Application Data
(60) Provisional application No. 60/967,949, filed on Sep. 7, 2007.

(51) Int. Cl. C07J 7/00

(52) U.S. Cl. 552/598

(57) ABSTRACT

The present invention provides for a crystalline polymorph of levonorgestrel and processes for making the same.

### BACKGROUND OF THE INVENTION

1. Field of the Invention

The present application claims priority to Provisional Patent Application 60/967,949, filed Sep. 7, 2007, and incorporates herein the entire disclosure of the provisional patent application.

### SUMMARY OF THE INVENTION

The present invention provides for a method of crystallizing levonorgestrel, and a novel crystalline polymorph of levonorgestrel.

### BRIEF DESCRIPTION OF THE DRAWINGS

FIG. 1 is the diffuse reflectance spectrum of the crystalline polymorph of levonorgestrel of the present invention.

FIG. 2 provides the peak positions within the diffuse reflectance spectrum of FIG. 1, as well as an expansion of a certain region of that spectrum.

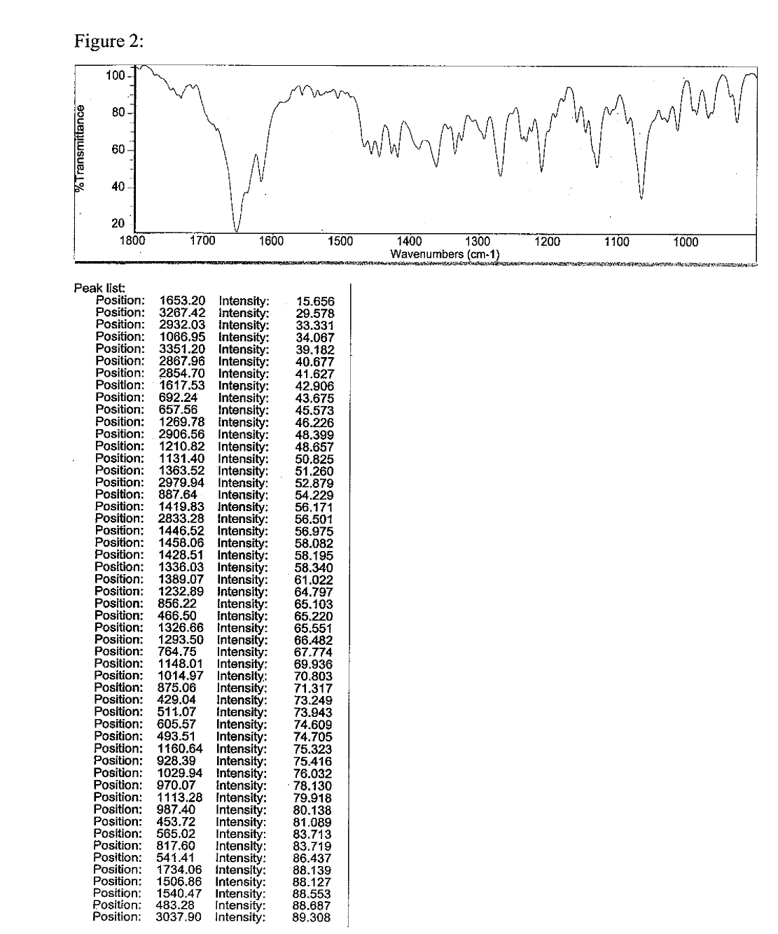

FIG. 3 provides an expansion of various regions of the diffuse reflectance spectrum of FIG. 1.

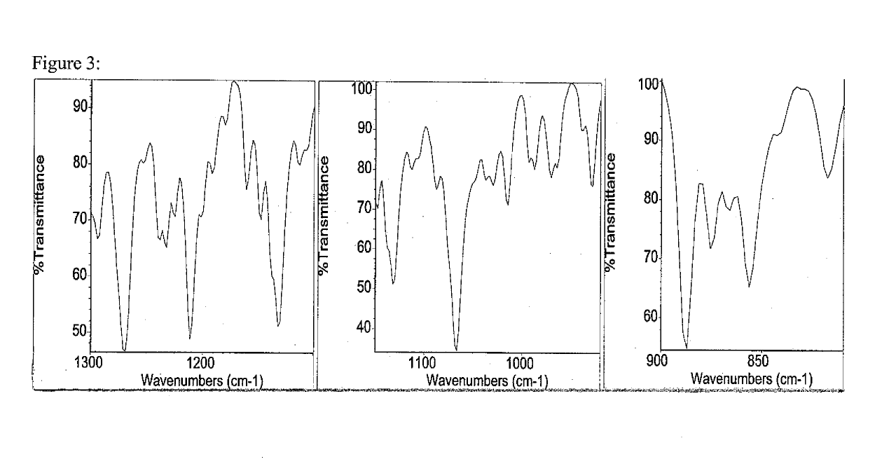

FIG. 4 shows the X-ray powder diffraction pattern of the crystalline polymorph of levonorgestrel of the present invention in intensity versus degrees in two theta.

FIG. 5 shows the X-ray powder diffraction pattern of the crystalline polymorph of levonorgestrel of the present invention in intensity versus d-spacing.

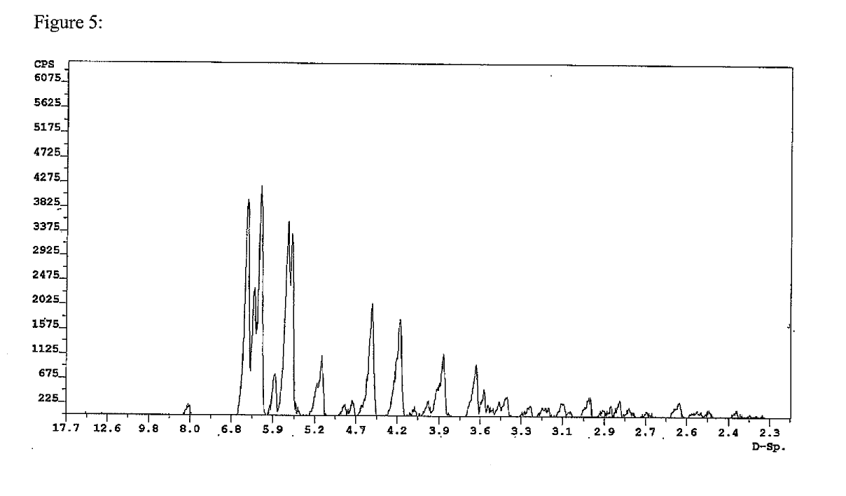

FIGS. 6 a and b list the peak positions and their intensities.

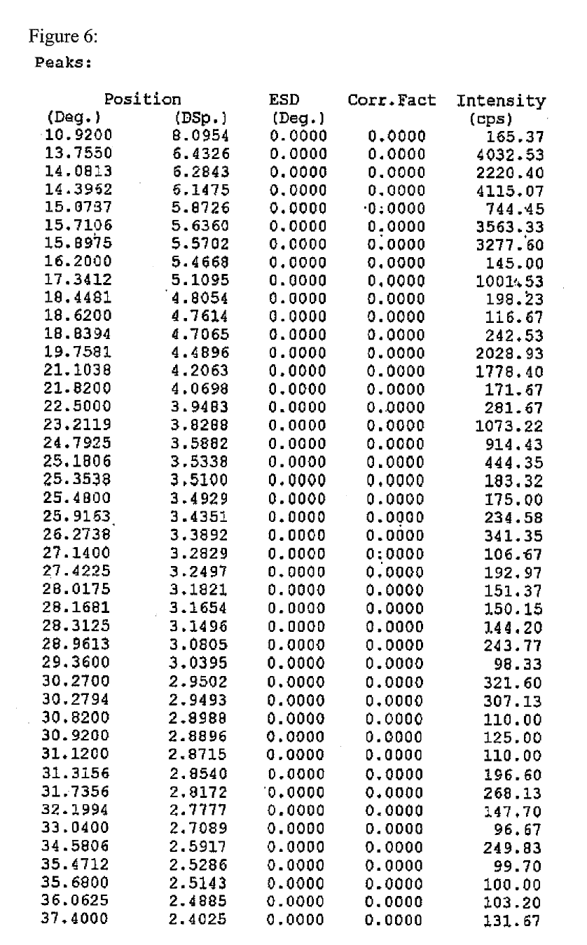

### DETAILED DESCRIPTION OF THE PRESENTLY PREFERRED EMBODIMENTS

The method of crystallizing the polymorph of the present invention is by the following procedures:

### (A) Ethyl Acetate/n-Heptane

To a suitable reactor is charged ethyl acetate (EA) (about 25 mL) and levonorgestrel (about 1 g). The slurry is stirred and heated to reflux (75˜78° C.) to form clear solution. n-Heptane (about 75 mL) is slowly charged and the solution is kept at 70˜75° C. The solution is cooled to 0˜5° C. in not less than (NLT) 90 minutes, and held for NLT 2 hours. The slurry is filtered and dried at 20˜30° C. to obtain about 0.5 g of Levonorgestrel.

### (B) Methanol/Water

Methanol (about 25 mL) is charged with Levonorgestrel (about 1 g). The slurry is stirred and heated to reflux (64˜65° C.) to form clear solution. Water (about 75 mL) is slowly charged and the solution is kept at 65˜70° C. The solution is cooled to 40˜45° C. in NLT 50 minutes, and hold for 1 hour. The slurry is filtered and dried at 20˜30° C. to obtain levonogestrel (about 0.6 g).

### (C) Methanol

Methanol (about 25 mL) is charged with Levonorgestrel (about 1 g). The slurry is stirred and heated to reflux (64˜65° C.) to form clear solution. The solution is cooled to 0˜5° C. in NLT 50 minutes, and hold for 1 hour. The slurry is filtered and dried at 20˜30° C. to obtain levonorgestrel (about 0.4 g).

Representative infrared spectrum and X-ray powder diffraction pattern for the crystalline levonorgestrel product are provided in the figures herein.

1. A crystalline polymorph of levonorgestrel exhibiting an X-ray powder diffraction pattern comprising a peak in degrees 2θ±0.2° 2θ at 13.8.

2. A crystalline polymorph of levonorgestrel according to claim 1 further comprising peaks in degrees 2θ±0.20° 2θ at 14.1 and 14.4.

3. The crystalline polymorph of levonorgestrel according to claim 1 further comprising peaks in degrees 2θ±0.2° 2θ at 15.7 and 15.9.

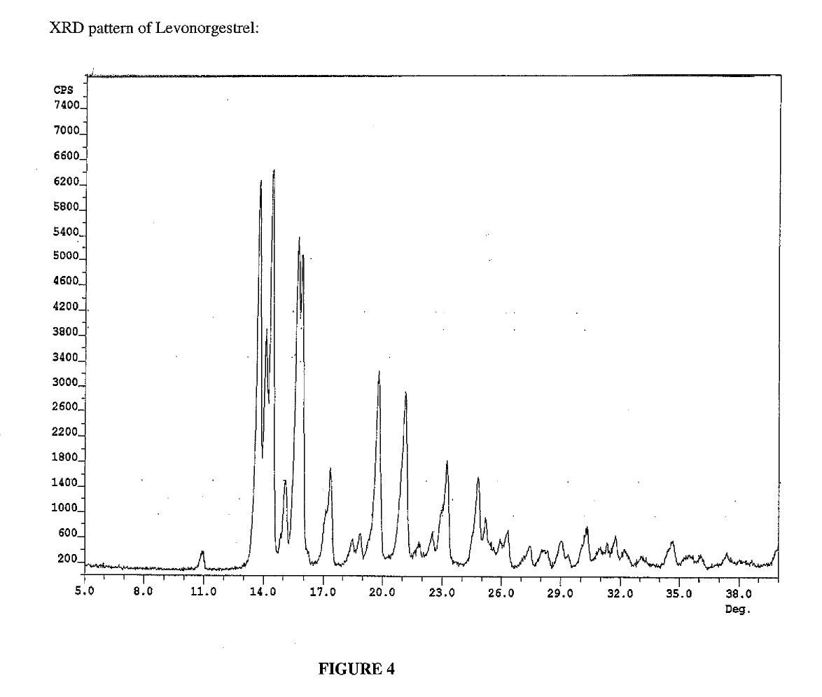

4. The crystalline polymorph of levonorgestrel according to claim 1 exhibiting an X-ray powder diffraction pattern substantially as shown in FIG. 4.

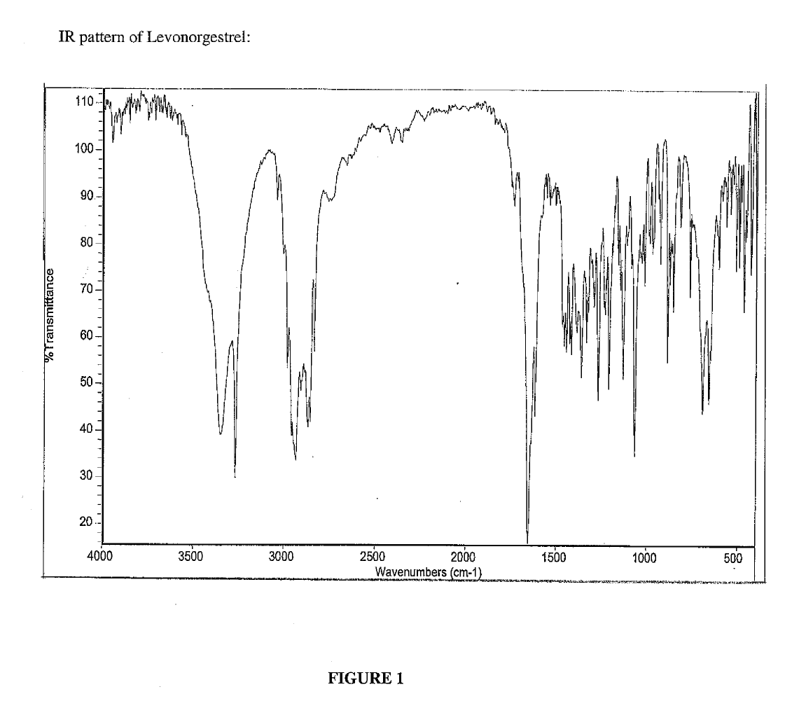

5. The crystalline polymorph of levonorgestrel according to claim 1 exhibiting a diffuse reflectance spectrum substantially as shown in FIG. 1.

6. A method of preparing a crystalline polymorph of levonorgestrel comprising the steps of: (a) dissolving levonorgestrel in ethyl acetate at an elevated temperature to form a solution; (b) adding n-heptane to the solution at the elevated temperature; (c) cooling the mixed solution to produce the crystalline polymorph of levonorgestrel.

7. A method of preparing a crystalline polymorph of levonorgestrel comprising the steps of: (a) dissolving levonorgestrel in methanol at an elevated temperature to form a solution; and (b) cooling the solution to produce the crystalline polymorph of levonorgestrel.

8. The method according to claim 7 further comprising the step of adding water to the solution at the elevated temperature after step (a) and before step (b).
